(19) United States
(12) Patent Application Publication
(10) Pub. No.: US 2022/0339470 A1
(43) Pub. Date: Oct. 27, 2022

(54) RESPIRATOR DEVICES WITH SENSORS, AND ASSOCIATED SYSTEMS AND METHODS

(71) Applicants: Hamish Jock GRAHAM, Auckland (NZ); O2-O2, Inc., Brooklyn, NY (US)

(72) Inventors: Daniel Bowden, Auckland (NZ); Ilya Rozhdestvensky, Auckland (NZ); Jeremy Mark Mauger, Auckland (NZ); Hamish Jock Graham, Auckland (NZ)

(21) Appl. No.: 17/642,627

(22) PCT Filed: Sep. 11, 2020
(86) PCT No.: PCT/US2020/050450

§ 371 Date: Mar. 11, 2022

(30) Foreign Application Priority Data
Sep. 12, 2019 (NZ) 757196

(51) Int. Cl. A62B 18/00; A61M 15/00; A62B 9/02; A62B 9/00; A62B 18/02

(52) U.S. Cl. CPC A62B 18/003; A61M 15/0001; A62B 9/022; A62B 9/006; A62B 18/025

(57) ABSTRACT

The present technology relates to open respirator devices having sensors for measuring breath biometrics. For example, a respirator device (100) can include a mask component (102) that fits at least partially over a user's face and forms a gap (112) between a periphery of the mask component and the user's face, thereby forming a breathing chamber (108) between the mask portion and the user's face. A fan unit can provide air from the external environment to the breathing chamber for the user to inhale. In some embodiments, the air may be filtered before entering the breathing chamber and the fans may provide a positive pressure seal between across the gap. One or more sensors can be coupled to the mask component for measuring one or more metrics associated with the user's breath.

### CROSS-REFERENCE TO RELATED APPLICATION

This application claims the benefit of New Zealand Provisional Patent Application No. 757196, filed Sep. 12, 2019, which is incorporated herein by reference in its entirety.

### TECHNICAL FIELD

The present technology generally relates to respirator devices and, in particular, to respirator devices with sensors.

### BACKGROUND

Air may contain particulates and/or pathogens that can adversely impact a person's health if inhaled. For example, air can contain varying amounts of dust, dirt pollen, smog, smoke, pollution, noxious fumes, toxic mists, and other respiratory irritants. These irritants can be dangerous if inhaled and may increase the likelihood of a person developing certain conditions, such as lung cancer and cardiovascular disease. In addition to respiratory irritants, air may also include infectious pathogens such as viruses and bacteria. For example, diseases that can be spread through inhaling airborne pathogens include COVID-19, anthrax, chickenpox, influenza, measles, smallpox, cryptococcosis, and tuberculosis.

Conventional face masks are frequently ineffective for protecting a user from inhaling airborne particulates and/or pathogens. Conventional face masks attempt to create a seal with a user's face to minimize the amount of atmospheric particulates and/or pathogens the user inhales. Some face masks incorporate filters or other features for filtering out particulates and/or pathogens. Additionally, conventional face masks are typically uncomfortable to wear, unaesthetic, interfere with communication, and place an extra respiratory burden on the user, which frequently leads to poor user compliance and respiratory distress. Accordingly, improved respirator devices capable of protecting a user from airborne particulates and pathogens are needed.

### BRIEF DESCRIPTION OF THE DRAWINGS

Many aspects of the present technology can be better understood with reference to the following drawings. The components in the drawings are not necessarily to scale. Instead, emphasis is placed on clearly illustrating the principles of the present technology.

FIG. 1A is an isometric view of a respirator device configured in accordance with embodiments of the present technology.

FIG. 1B is a top view of the respirator device of FIG. 1A.

FIG. 1C is a front view of the respirator device of FIG. 1A.

FIG. 1D is a back view of the respirator device of FIG. 1A.

FIG. 1E is a side view of the respirator device of FIG. 1A.

FIG. 1F is a box diagram of select components of the device of FIG. 1A configured in accordance with embodiments of the present technology.

FIG. 2A is a partially schematic top view of the device of FIG. 1A during an inhalation phase of a user breath in accordance with embodiments of the present technology.

FIG. 2B is a partially schematic top view of the device of FIG. 1A during an exhalation phase of a user breath in accordance with embodiments of the present technology.

FIG. 3 is a flowchart of a method for determining a corrected value of a measured breath metric in accordance with embodiments of the present technology.

FIG. 4 is a flowchart of a method for calculating a corrective factor for correcting a measured value of a breath metric in accordance with embodiments of the present technology.

FIG. 5 is a look-up-table for determining a corrective factor based on a pressure value and a filtered air supply rate in accordance with embodiments of the present technology.

FIG. 6 is a flowchart of a method for measuring breath metrics in accordance with embodiments of the present technology.

### DETAILED DESCRIPTION

### A. Overview of the Technology

The present technology is generally directed to respirator devices, and associated systems and methods. In some embodiments, a respirator device (also referred to herein as a “respiratory mask,” “respirator system,” or “device”) is worn by a user. The respirator device can be an “open” device that does not form a contact seal with the user's face. For example, the respirator device can include a mask component (also referred to herein as a “mask region,” “mask section,” or “mask portion”) that fits at least partially over (e.g., in front of) a user's skin and is sized and shaped to form one or more gaps spacing a periphery of the mask component apart from the user's face, thereby forming a breathing chamber between the mask component and the user's face that supplies air for inhalation and receives exhaled air. The respirator device can include a fan unit that provides filtered air from the surrounding environment to the breathing chamber for the user to inhale. In various embodiments, the fan unit may maintain a positive pressure within the breathing chamber to provide a non-contact positive pressure seal on the mask. The respirator device can also include one or more sensors coupled to the mask component and/or in communication with the breathing chamber to measure one or more metrics associated with the user's breath.

In some embodiments, the user's exhalation gases mix with the filtered incoming air in the breathing chamber. Thus, the user's exhalation gases are diluted by the filtered air before reaching the one or more sensors. The present technology thus provides systems and methods that can adjust a value of a measured breath metric to correct for the dilution of the user's exhalation gases that occurs in the breathing chamber. For example, in some embodiments the present technology includes a respiratory analysis module that determines a corrective factor (also referred to as a “dilution factor”) corresponding to a ratio of exhalation gases to filtered air in the breathing chamber, and applies the corrective factor to the measured breath metric to correct for the dilution of the user's exhalation gases. Thus, without being bound by theory, the present technology enhances the accuracy of sensed breath biometrics in open respirator devices.

The present technology is expected to address many shortcomings of other face masks that include breath sensors. For example, while seal-based masks can incorporate sensors for measuring aspects of breath biometrics, the tight fit and constant contact often make them uncomfortable, provide breathing resistance, and induce rebreathing of previously exhaled air. They may also affect the accuracy of any breath biometrics measured using sensors coupled to the masks. For example, the user must forcefully inhale against the pressure drop of the air passing through a filter media, and then must forcefully exhale to force the air out through an exhaust valve. This can impact the user's ability to breath normally, which may impact the resultant respiratory state and thus the user's breath characteristics. Moreover, the accuracy of the sensors is dependent upon achieving a substantially airtight seal with the user's face, which can be difficult given the variability in facial structure.

The present technology provides face masks and other respirator devices that can accurately and comfortably measure breath biometrics, while also providing filtered clean air for breathing. For example, the technology described herein can include numerous advantages over other face masks in that it can accurately measure breath biometrics while also providing other advantages, such as one or more following: improved comfort for the user as it is lightweight; has an engineered gap for breathing assistance or to enable natural breathing conditions (e.g., to relieve the user from expending more energy breathing relative to a closed system); is battery-powered, hence portable; and/or uses a clear shield that allows for empirical observation of a user's breathing conditions and health (e.g., color of the lips) and removes the visual barrier to communicate with others.

Further aspects and advantages of the devices, methods, and uses will become apparent from the ensuing description that is given by way of example only.

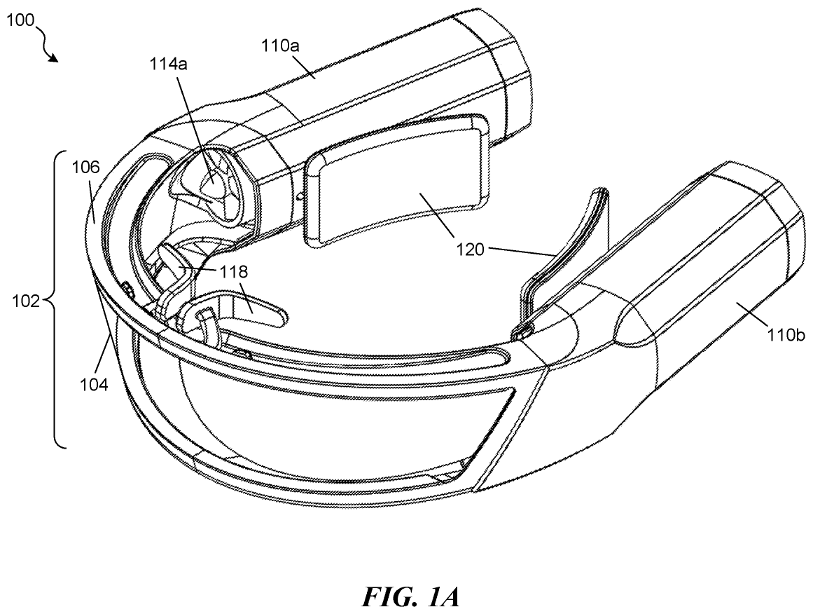

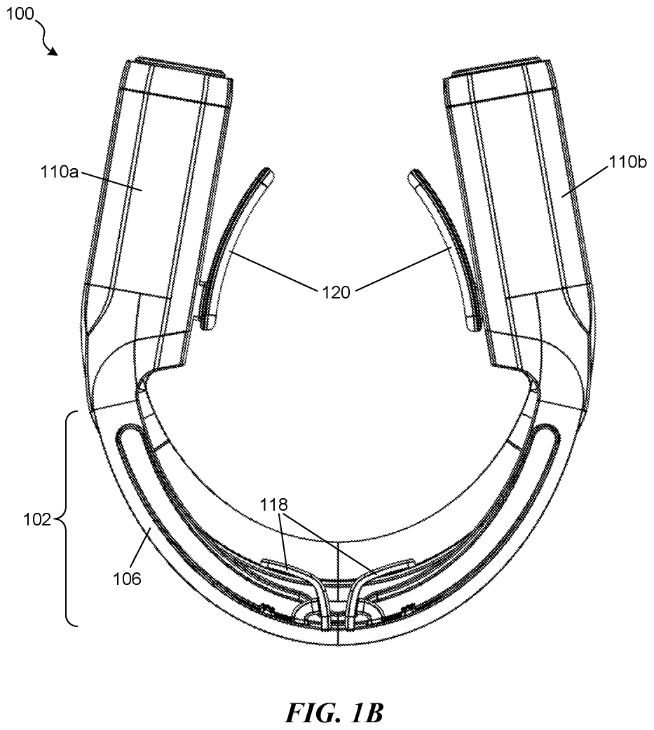

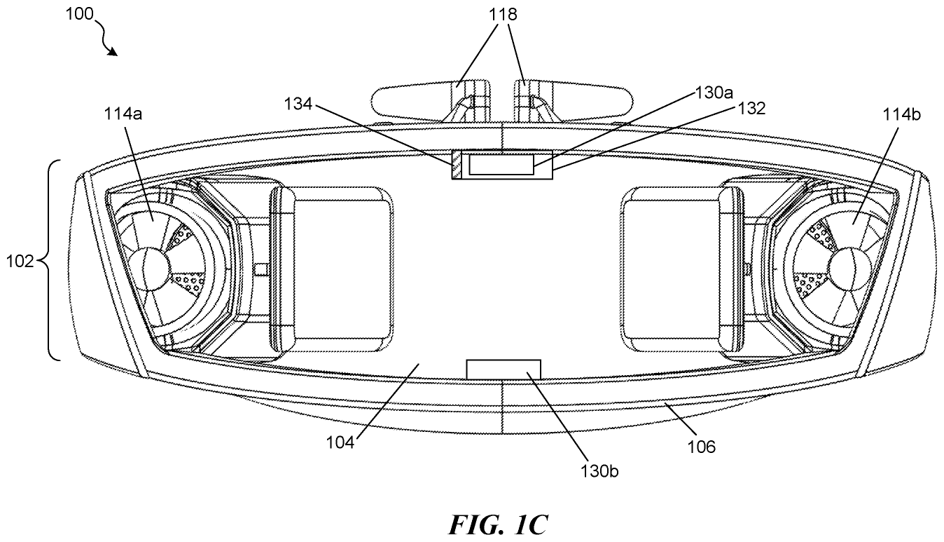

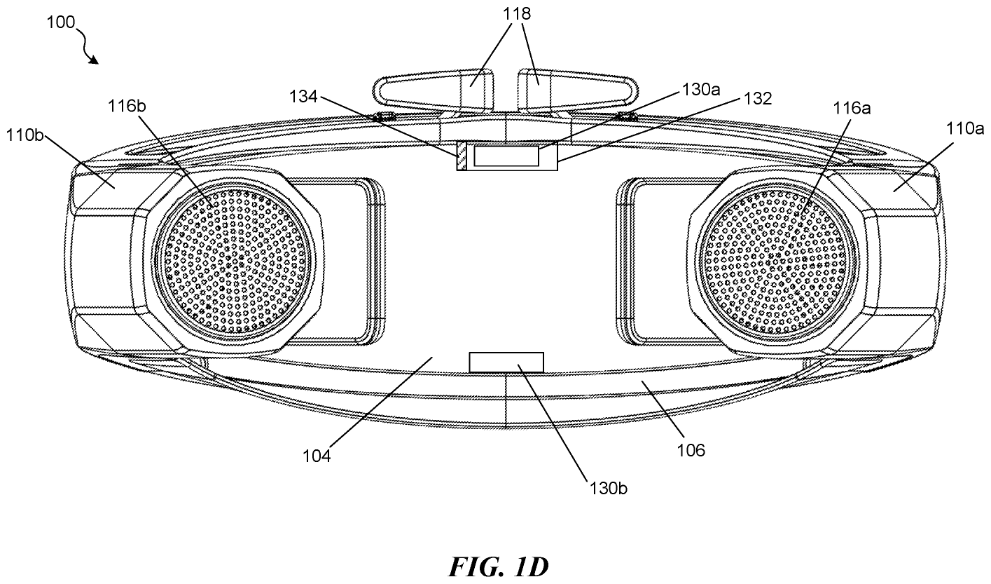

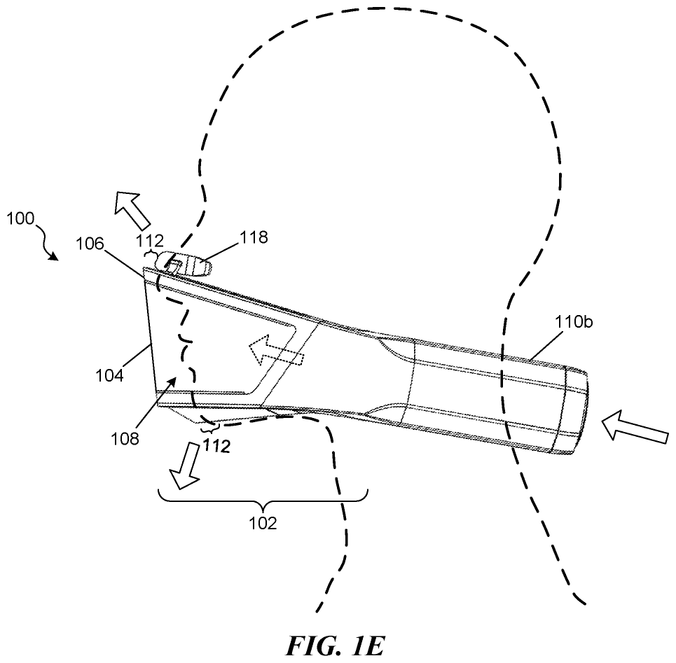

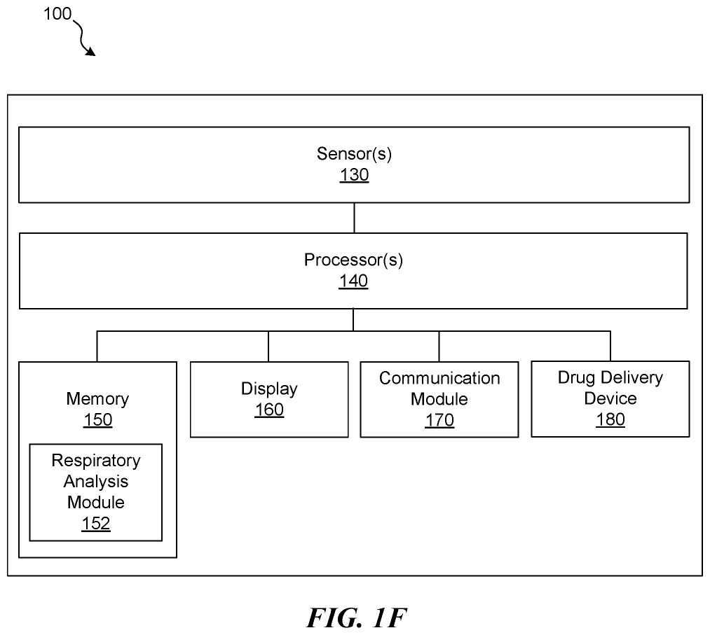

The terminology used in the description presented below is intended to be interpreted in its broadest reasonable manner, even though it is being used in conjunction with a detailed description of certain specific embodiments of the present technology. Certain terms may even be emphasized below; however, any terminology intended to be interpreted in any restricted manner will be overtly and specifically defined as such in this Detailed Description section. Additionally, the present technology can include other embodiments that are within the scope of the examples but are not described in detail with respect to FIGS. 1A-6.

Reference throughout this specification to “one embodiment” or “an embodiment” means that a particular feature, structure, or characteristic described in connection with the embodiment is included in at least one embodiment of the present technology. Thus, the appearances of the phrases “in one embodiment” or “in an embodiment” in various places throughout this specification are not necessarily all referring to the same embodiment. Furthermore, the particular features or characteristics may be combined in any suitable manner in one or more embodiments.

Reference throughout this specification to relative terms such as, for example, “generally,” “approximately,” and “about” are used herein to mean the stated value plus or minus 10%. The term “substantially” or grammatical variations thereof refers to at least about 50%, for example, 75%, 85%, 95%, or 98%.

The headings provided herein are for convenience only and do not interpret the scope or meaning of the claimed present technology.

### B. Select Embodiments of Respirator Systems and Devices

FIGS. 1A-1F and the accompanying description provide a general overview of a representative respirator system or device 100 (hereinafter “device 100”) configured in accordance with embodiments of the present technology. More specifically, FIG. 1A is an isometric view of the device 100, FIG. 1B is a top view of the device 100, FIG. 1C is a front view of the device 100, FIG. 1D is a back view of the device 100, FIG. 1E is a side view of the device, and FIG. 1F is a box diagram of select components of the device 100.

Referring to FIGS. 1A-1E together, the device 100 includes a mask component 102 (also referred to as a “mask section”) configured to partially or fully cover a user's face (e.g., a user's nose and/or mouth; best seen in FIG. 1E). The mask component 102 can have an elongated, curved shape that extends at least partially around and generally corresponds to the curvature of a human face. The mask component 102 can include a shield 104 configured to block air flow to and/or from the user's face. The shield 104 can be made out of any suitable air-impermeable material, such as plastic. For example, the shield 104 can be made of a thin plastic sheet or film, e.g., to reduce weight and/or improve sound transmission. For example, the shield 104 can be no more than 5 mm, 4.5 mm, 4 mm, 3.5 mm, 3 mm, 2.5 mm, 2 mm, 1.5 mm, 1 mm, 0.75 mm, 0.5 mm, 0.25 mm, or 0.1 mm in thickness. In the illustrated embodiment, the shield 104 is transparent, wherein in other embodiments the shield 104 can be opaque, partially opaque, or adjustable in transparency. One advantage of a transparent shield is that the user's face can be easily seen and monitored. For example, healthcare personnel or other individuals may monitor the user's face for visual indicators of the health condition such as pain, confusion, and/or vital signs (e.g., lip color). Additionally, a transparent shield can reduce interference with communication when wearing the device 100. In other embodiments, however, the shield 104 can be translucent, opaque, or adjustable in transparency.

The shield 104 can be coupled to (e.g., mounted in or otherwise attached to) a frame 106. In the illustrated embodiment, the frame 106 surrounds the entire perimeter of the shield 104 (best seen in FIG. 1C). In other embodiments, the frame 106 can extend only around a portion of the perimeter of the shield 104 (e.g., only the top and bottom edges, only the lateral edges, etc.). The frame 106 can provide mechanical support to the shield 104, and can also serve as a connection point to other components of the device 100. In embodiments in which the shield 104 is made of a relatively thin material (e.g., a thin plastic film), the frame 106 can be made of a rigid material that holds the shield 104 in tension in order to maintain its shape. As best shown in FIG. 1E, in some embodiments the shield 104 and the frame 106 are at least partially spaced apart from the user (e.g., the frame 106 and the shield 104 do not directly contact the user when the device 100 is worn), creating one or more gaps 112. As described in greater detail below, the airflow within the mask component 102 is controlled through the use of a pressure gradient, as described below.

As shown in FIG. 1E, the mask component 102 defines and partially surrounds a breathing chamber 108 (also referred to as “plenum 108”). When the device 100 is worn by the user, the breathing chamber 108 is partially enclosed on one side by the internal surface of the shield 104 (i.e., the surface of the shield 104 facing the user) and on another side by the user's face. Accordingly, the user inhales air from the breathing chamber 108 and exhales air into the breathing chamber 108. In various embodiments, the device 100 is sized and shaped such that a peripheral region of the mask component 102, which can be defined by portions of the shield 104 and/or the frame 106, is spaced apart from the user's face by one or more gaps 112 (also referred to as “openings,” “channels,” “slots,” and “vents”). The gaps 112 can each be approximately 0.25 cm, 0.5 cm, 0.75 cm, 1 cm, 1.25 cm, 1.5 cm, 2 cm, 2.5 cm, 3 cm, 3.5 cm, 4 cm, 4.5 cm, or 5 cm wide. Additionally, although FIG. 1E illustrates the gaps 112 as being at the upper and lower edges of the mask component 102, this configuration can be modified as desired (e.g., a single gap 112 at the upper edge, a single gap 112 at the lower edge, etc.). In some embodiments, the device 100 is configured so that the air follows a defined air flow path through the breathing chamber, e.g., air enters the breathing chamber 108 from a specific air intake region and exits the breathing chamber 108 through a specific air outflow region. For example, air from the external environment can enter the breathing chamber 108 through lumens within one or more elongated arm members 110a-b (also referred to as “arms”) coupled to one or both sides of the mask component 102 (FIGS. 1A and 1B), and can exit through the gaps 112 between the mask component 102 and the user's face. In other embodiments, however, the device 100 can have a different air flow path. For example, the air flow path shown in FIG. 1E can be reversed such that air enters the breathing chamber 108 through the gaps 112 and exits through the arms 110a-b.

The device 100 can include one or more fan units 114a-b (FIGS. 1A and 1C) to produce and/or maintain the air flow path through the breathing chamber 108. In the illustrated embodiment, the fan units 114a-b are mounted at least partially within the arms 110a-b, adjacent or near the breathing chamber 108. In other embodiments, however, the fan units 114a-b can be located at other portions of the device 100, as discussed in greater detail below. The fan units 114a-b can each be or include an impeller, rotor, or any other component configured to produce air flow. In operation, the fan units 114a-b can draw in or otherwise move air (also referred to herein as “external air,” “atmospheric air,” or “intake air”) from an environment external to the device 100 toward and/or to the breathing chamber 108. In some embodiments, the fan units 114a-b are each configured to produce an air flow rate greater than or equal to 10 L/min, 20 L/min, 30 L/min, 40 L/min, 50 L/min, 60 L/min, 70 L/min, 80 L/min, 90 L/min, 100 L/min, 125 L/min, 150 L/min, 175 L/min, 200 L/min, 225 L/min, 250 L/min, 275 L/min, or 300 L/min. The air flow rate may be varied, e.g., by changing the rotation speed of the fan units 114a-b.

Optionally, the fan units 114a-b can also be configured to maintain a desired pressure level in the breathing chamber 108 when the device 100 is worn. For example, the fan units 114a-b can be configured to maintain a positive pressure relative to the external environment, e.g., a positive pressure greater than or equal to 1 Pa, 2 Pa, 3 Pa, 4 Pa, 5 Pa, 6 Pa, 7 Pa, 8 Pa, 9 Pa, 10 Pa, 15 Pa, 20 Pa, 25 Pa, 30 Pa, 35 Pa, or 40 Pa. In some embodiments, the fan units 114a-b are configured to maintain a positive pressure relative to the external environment within a range from 5 Pa to 10 Pa during normal operation (e.g., while the user is exhaling at a normal rate), and within a range from 20 Pa to 30 Pa while the user is exhaling at a higher than normal rate (e.g., due to coughing, sneezing, heavy breathing, etc.). This can create a positive pressure seal that does not allow air from the external environment to enter through the gaps 112, thereby only allowing the user to breath filtered air entering the breathing chamber via the fan units 114, while still permitting exhaled air to exit via the gaps 112. In various embodiments, the fan units 114a-b can be configured to maintain a negative pressure relative to the external environment, e.g., a negative pressure less than or equal to −1 Pa, −2 Pa, −3 Pa, −4 Pa, −4.5 Pa, −5 Pa, −5.5 Pa, −6 Pa, −6.5 Pa, −7 Pa, −8 Pa, −9 Pa, −10 Pa, −15 Pa, −20 Pa, or −25 Pa. This provides a negative pressure seal that allows external air in through the gaps 112, but maintains the exhaled air within the breathing chamber such that the exhaled air does not enter the external environment.

The device 100 can also include components that filter (e.g., sterilize, sanitize, etc.) the air entering and/or exiting the breathing chamber 108 to protect the user from irritants, pollutants, and/or infectious agents in the external environment and/or to protect others from infectious agents exhaled by the user. For example, the device 100 may filter the atmospheric air being drawn into the system to provide filtered atmospheric air to the breathing chamber 108. In some embodiments, the device 100 is configured to remove and/or inactivate at least 75%, 80%, 90%, 95%, 99%, 99.5%, 99.9%, 99.99%, or 99.999% of the irritants, pollutants, and/or infectious agents entering and/or exiting the breathing chamber 108. The filtering mechanisms of the device 100 can be configured in many different ways. For example, the arms 110a-b can each include one or more filters 116a-b configured to obstruct pathogens from entering the breathing chamber 108.

The device 100 may also include one or more sensors (e.g., a first sensor 130a and a second sensor 130b, shown in FIGS. 1C and 1D, and collectively referred to as “the sensors 130”). Although shown as including two sensors 130, the device 100 can include fewer or more sensors 130, such as one, three, four, five, six, seven, eight, or more. The sensors 130 can detect and monitor the operation of the mask (e.g., pressure levels, air flow, temperature, air quality), the user's condition (e.g., breath metrics, body temperature, respiratory rate, pulse, blood pressure), and/or user compliance (e.g., whether the user is wearing the device properly). Examples of sensors suitable for use within the devices described herein therefore include, but are not limited to: breath sensors, particle sensors, pressure sensors, moisture sensors, flow sensors, temperature sensors, infrared sensors, air quality sensors, blood pressure sensors, heart rate sensors, blood oxygenation sensors, audio sensors, image sensors, light sensors, accelerometers, gyroscopes, global positioning systems, and/or combinations thereof.

In some embodiments, at least one of the sensors 130 is a breath sensor that measures one or more metrics associated with a user's exhalation gases (also referred to herein as “breath metrics” or “breath biometrics”). The breath sensor can measure a concentration (e.g., ppm, percentage, etc.) of particulates, gases, or other compounds in the user's exhalation gases, including, for example, volatile organic compounds (VOC) (e.g., isoprene, acetone, etc.), oxygen (O₂), carbon dioxide (CO₂), hydrogen (H₂), nitrogen (N₂), nitric oxide (NO), nitric dioxide (NO₂), total nitric oxides (NOₓ), sulphur oxides (SOₓ), argon (Ar), water vapor, and/or ammonia. In some embodiments, the breath sensor can detect the presence of one or more target bacteria and/or viral material in a user's exhalation breath. In some embodiments, the breath sensor can measure other metrics associated with a user's breath, such as respiratory rate, respiratory volume, respiratory type, humidity level, breath temperature, VO₂ max, and the like. In some embodiments, the sensors 130 can measure other metrics associated with the user, such as positional information, acceleration, speed, and the like.

In embodiments in which the sensors 130 measure a metric associated with the user's breath, the sensors 130 are configured to receive (i.e., be exposed to) at least a portion of exhaled breath. For example, the sensors 130 can be coupled to an interior surface of the shield 104 and/or the frame 106 such that they are positioned within and/or in fluid communication with the breathing chamber 108. In the illustrated embodiment, for example, the first sensor 130a is positioned at an upper portion of the shield 104 and the second sensor 130b is positioned at a lower portion of the shield 104. The sensors 130 are also positioned in a generally central portion of the shield along its width such that, when the device 100 is worn by a user, the sensors 130 are directly (or at least generally directly) in front of the user's mouth. In some embodiments, the sensors 130 include a sensor body and a sensing element or probe operably coupled to but distinct from the sensor body. In such embodiments, the sensing element can positioned generally directly in front of the user's mouth, while the sensor body can be coupled to a different portion of the device 100 (e.g., positioned within or along the arms 110a-b).

In some embodiments, one or more of the sensors 130 can be positioned within a housing 132 or other structure that fluidically isolates the sensors 130 from the breathing chamber 108. Although only the first sensor 130a is shown in the housing 132 in FIGS. 1D and 1E, in some embodiments more than one sensor may be positioned in the housing 132, and/or the device 100 can include multiple discrete housings 132 for different sensors 130, such as a first housing for the first sensor 130a and a second housing for the second sensor 130b. The housing 132 can have a moveable member 134, such as a door or valve, that can move between an open position and a closed position to expose a sensing component to the breathing chamber. For example, the moveable member 134 can be a piezoelectric valve, a MEMs valve, a gate valve, or any other suitable valve. When the valve is in the open position, the interior of the housing 132 (and thus the sensors 130) can be in fluid communication with the breathing chamber 108. When the moveable member 134 is in the closed position, the interior of the housing (and thus the sensors 130) can be fluidly isolated from the breathing chamber 108. In operation, the moveable member 134 can move from the closed position to the open position (or remain in the open position) when a sensor measurement is desired, and move from the open position to the closed position (or remain in the closed position) when a sensor measurement is not desired. This enables the sensors 130 to selectively measure the breath metrics during select phases of the user's breathing (e.g., during an exhalation phase, during an inhalation phase, while holding breath), which can be useful when calibrating the sensors 130 over multiple breathing cycles. In some embodiments, the moveable member 134 is generally open during the exhalation phase of a user's breath and closed during an inhalation phase of the user's breath.

In some embodiments, the sensors 130 continuously measure and record values of various metrics associated with the user. In some embodiments, the sensors 130 intermittently or periodically measure and record the various metrics associated with the user. For example, in some embodiments the sensors 130 may measure and record the metrics multiple times per second, such as 20 times per second, 15 times per second, 10 times per second, 5 times per second, or 2 times per second. In some embodiments, the sensors 120 may measure and record the metric once per second or once every 2 seconds, 5 seconds, 10 seconds, 20 seconds, 30 seconds, 60 seconds, 2 minutes, 5 minutes, 10 minutes, 30 minutes, 60 minutes, etc. In some embodiments, the sensors 130 measure and record values of the metrics during select phases of the user's respiratory cycle. For example, the sensors 130 can be automatically activated during an exhalation phase of the user's breath (e.g., to capture breath biometrics) and inactivated during an inhalation phase of the user's breath and/or between inhalation and exhalation phases. In some embodiments, the sensors 130 are continuously active but are positioned within the housing. In such embodiments, even though the sensors 130 are continuously measuring various metrics, the housing valve can be operated such that the sensors 130 are in fluid communication with the breathing chamber 108 during the exhalation phase but fluidly isolated from the breathing chamber 108 during other phases of the breath. Without being bound by theory, using a valve to control timing of the sensor measurements rather than directly controlling the sensor itself (e.g., turning it off and on) is expected to increase the ability to match the measurements with the target breathing phase. In some embodiments, the device 100 can have a control module (e.g., an application on a smart phone) from which a user can direct one or more of the sensors 130 to measure various metrics (e.g., “on demand” measurements).

FIG. 1F is a box diagram illustrating additional select components of the device 100. As illustrated, in some embodiments the device 100 can include a processor 140. The processor 140 can be, for example, a conventional microprocessor such as an Intel Pentium microprocessor or Motorola power PC microprocessor. The device 100 can further include a memory 150. The memory 150 can be coupled to the processor by, for example, a bus (not shown). The memory 150 can include, by way of example but not limitation, random access memory (RAM), such as dynamic RAM (DRAM) and static RAM (SRAM). The memory 150 can be local, remote, or distributed. Various values recorded by the sensor 130 can be stored in the memory 150. For example, the sensor 130 may continuously or intermittently record values associated with a breath biometric, and the device 100 may store the recorded values in the memory 150. The memory 150 can also store various software modules, such as a respiratory analysis module 152.

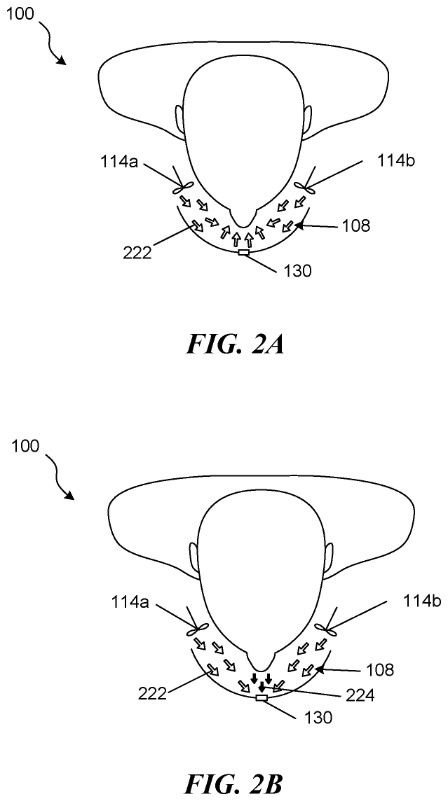

The respiratory analysis module 152 can include computer-executable instructions that, when executed, cause the device 100 to execute any of the methods described herein. For example, as will be described in greater detail with respect to FIGS. 2A-6, the respiratory analysis module 152 can include instructions that, when executed, determine a “corrective factor” that can be used to adjust a value measured by the sensor 130 to provide a corrected value that accounts for a dilution of the exhalation gases that occurs as they mix with the filtered atmospheric air in the breathing chamber 108. Accordingly, as will be described in greater detail below, the respiratory analysis module 152 can perform any of the calculations described herein for determining and/or applying the corrective factor to various recorded breath metrics. The output from the respiratory analysis module 152 can therefore be a corrective factor, an adjusted breath metric, or both a corrective factor and an adjusted breath metric.

In addition to determining and/or applying a corrective factor, the respiratory analysis module 152 can also analyze the values of the metrics recorded by the sensors 130 to determine additional information about the user. In a first example, the sensors 130 can include a humidity sensor, and the respiratory analysis module 152 can analyze the measured humidity values to determine whether the user was breathing through his/her nose or mouth during the recording. In such embodiments, a baseline humidity measurement of the filtered atmospheric air in the breathing chamber 108 can be recorded and provided to the respiratory analysis module 152. The respiratory analysis module 152 can then compare subsequent humidity measurements taken during an exhalation phase to the baseline value. In some embodiments, the humidity sensor is enclosed in a housing with a valve (e.g., as previously described with respect to FIG. 1), and the valve is only open during the exhalation phase of the breath. Without being bound by theory, the humidity in the exhalation gases is expected to be higher when the user is breathing through their mouth than when the user is breathing through their nose. Therefore, the respiratory analysis module 152 can determine whether the user is breathing through their nose or their mouth based on the relative change in humidity from the baseline value. In a second example, the sensors 130 can include a microphone for recording sounds, and the respiratory analysis module 152 can analyze the recorded sounds to determine whether the user was breathing through his/her nose or mouth based on the recording. In such embodiments, the respiratory analysis module 152 can include filters for excluding undesired sounds, such as the operational sounds of the fans 114.

In some embodiments, the respiratory analysis module 152 can store a set of parameters associated with an individual user and/or average metrics associated with a population of users. During operation, the respiratory analysis module 152 can compare metrics measured by the sensors (either before or after the metrics are adjusted for dilution, as described below with respect to FIGS. 2A-6) and provide alerts to the user if the measured metrics exceed or fall beneath a predetermined threshold. For example, the respiratory analysis module 152 can include a range of CO₂ that is standard in exhalation gases (e.g., between about 4.0-5.3% by volume of exhalation gases). If the measured CO₂ value exceeds the range, the respiratory analysis module 152 can generate an alert, which can be a visual alert (e.g., lighting, text, etc.), an audible alert (e.g., tones, spoken instructions), and/or haptic alert (e.g., buzzing, vibration, etc.). As another example, the respiratory analysis module 152 can generate an alert if an acetone concentration in the exhalation gases exceeds a predetermined ratio, such as 0.01%. Alerts can also be communicated to a display on the device (e.g., display 160, described below) or to a separate device (e.g., a device carried by the user or another individual, such as a smartphone, smartwatch, tablet, laptop computer, personal computer, etc.) via wired or wireless communication methods (e.g., Bluetooth).

In some embodiments, the device 100 includes a display 160 used to display various types of output, such as text and/or graphical models of the metrics recorded by the sensor 130 and/or alerts generated by the respiratory analysis module 152. The display 160 can be an LED display screen, an LCD display screen, an augmented reality display, or the like. The display 160 can be physically coupled to a portion of the device 100 adjacent the user's face (e.g., the frame 106) such that the display 160 is in the user's field of vision when the user is wearing the device 100. In other embodiments, the display 160 is not incorporated into the device, but rather is a separate device that is operably coupled to (e.g., wirelessly coupled via Bluetooth, Wireless Fidelity (WIFI), or the like) to the device 100. For example, the display 260 can be a smart-phone screen, a smart-watch screen, a computer screen, a tablet screen, or the like.

In some embodiments, the device 100 includes a communication module 170 used to transmit data from the device 100, such as values of the metrics recorded by the sensor 130. The communication module 170 can be a wireless transceiver configured to transmit and/or receive wireless signals for communicating information between the device 100 and another linked device or system (e.g., a mobile phone, a smart watch, a tablet, a computer, etc.). The wireless communications and the corresponding transceiver circuits can be implemented according to a predetermined protocol or a standard, such as for Bluetooth, Near Field Communication (NFC), and/or WIFI. In some embodiments, the wireless communication link can provide a direct link (e.g., without any intervening devices) between the connected devices. Further, the wireless communication link can be implemented for devices located within a relatively short distance threshold, such as for Bluetooth and NFC in comparison to other communication technologies (e.g., cellular communication technology). In some embodiments, the communication module 170 can be configured to transmit data to one or more remote analysis devices or networks for collecting and analyzing user data, such as those described in New Zealand Provisional Patent Application No. 757199, filed Sep. 12, 2019, the disclosure of which is incorporated by reference herein in its entirety.

In some embodiments, the device 100 further includes a drug delivery device 180, such as a nebulizer. The drug delivery device 180 can be operably coupled to the sensors 130 (e.g., via the processor 140 and one or more drug delivery control modules stored in the memory 150), and can provide a predetermined amount of a drug or other therapeutic agent to the user based at least in part on the metrics recorded by the sensor 130. For example, if a recorded metric (after being adjusted for dilution) exceeds or falls below a predetermined threshold, the drug delivery device 180 can automatically deliver a predetermined amount of the drug to the user. In some embodiments, if the recorded metric exceeds or falls below the predetermined threshold, the device provides an alert to the user (e.g., via the display 160) asking if the user would like to receive the drug. The user can then indicate, via a user input (e.g., a microphone, a touch screen, etc.) whether they would like to receive the dose. The drug delivery device 130 can be coupled to the frame 106 or another portion of the device 100 such that an aerosolized drug (e.g., albuterol) mixes with the filtered atmospheric air in the breathing chamber 108 before being inhaled by the user.

As an example, the sensors 130 can be configured to detect an impending and/or ongoing asthmatic event (e.g., by measuring breathing patterns, breath sound, breath volume, etc.), and the drug delivery device 180 may include a reserve store of albuterol. If, based on the measured breath metrics, the respiratory analysis module 152 determines that the user is about to suffer or is suffering an asthmatic event, the drug delivery device 180 may automatically disperse a dose of albuterol to the user to mitigate and/or treat the asthmatic event. As another example, the sensors 130 may be configured to detect ketones in the breath, the presence of which may indicate the user has diabetic ketoacidosis. In response to detecting ketones, the drug delivery device 180 may deliver a therapeutic agent (e.g., insulin) to the user, and/or may provide an alert to the user to seek immediate medical attention. As one skilled in the art will appreciate, the foregoing examples are provided to more clearly illustrate the principles of the present technology, and in no way limit the scope of the present technology.

The device 100 can include additional functional components. For example, as best shown in FIG. 1A, the device 100 can include components for securing the device 100 to the user's body, such as a nosepiece 118 and/or neck pads 120. The nosepiece 118 and/or neck pads 120 can be adjustable to accommodate the particular user's anatomy. The device 100 can also include electronic components (not shown), such as a power source (e.g., a rechargeable or non-rechargeable battery), an interface for connection to an external power source, controllers (e.g., for the fan units 114a-b, power source, and/or other components of the device 100), and the like. The electronic components can be located on or within any suitable portion of the device 100. For example, some or all of the electronic components can be housed within one or both of the arms 110a-b. The mask component 102 may also include one or more openings to allow administration of medicines, drinks, and the like. The opening can be sufficiently large to allow a drinking straw, pills, and/or other medicines to be administered to the user, yet not too large such that the device can no longer maintain the desired pressure level.

In some embodiments, the device 100 is a lightweight, portable device that can be worn for extended period of time with little or no user discomfort. For example, the total weight of the device 100 can be less than or equal to 500 g, 400 g, 300 g, 200 g, or 100 g. The device 100 can be configured to operate continuously for at least 1 hour, 2 hours, 3 hours, 4 hours, 5 hours, 6 hours, 7 hours, 8 hours, 9 hours, or 10 hours without recharging.

In some embodiments, the device 100 is an athlete monitoring device that can be worn by an athlete during an athletic activity. Because the device 100 is lightweight and is generally spaced apart from the user's mouth and nose, the device 100 is not expected to interfere with the athlete's performance. The device 100 can therefore be used to monitor various breath and other metrics of the athlete during the athletic activity. Further, because the device 100 is portable, the athlete's breath metrics can be measured in a real-world, athletic environment (e.g., outdoors, playing a team sport, moving around a court, field, or ice), rather than being connected to wires and other devices while using a stationary exercise device. When being used as an athlete monitoring device, it may not be necessary to provide the filtered air supply, and instead the device 100 may be used solely for its sensing capabilities.

In some embodiments, the device 100 is designed to be disinfected and reused, e.g., using cleaning and/or sterilization techniques known to those of skill in art such as applying disinfectant, heat treatment, washing, etc. Optionally, the device 100, or individual components thereof (e.g., mask component, shield, frame, etc.) can include an antimicrobial surface, coating, or material to further reduce the risks of transmission when worn, donning, doffing, disinfecting, or otherwise being handled. In other embodiments, however, the device 100 can be designed to be single-use, disposable devices.

The above description of the device 100 is provided merely for illustrative purposes and is not intended to be limiting. For example, in some embodiments, one or more components of the device 100 may be omitted. Additionally, the device 100 may be modified to include additional elements not shown in FIGS. 1A-1F. Additionally, the present technology is not limited to the device 100 illustrated in FIGS. 1A-1F. For example, the present technology can be implemented in many different types of respirator devices, such as those described in PCT Patent Publication No. 2017/065620, filed Oct. 17, 2016, the disclosure of which is incorporated by reference herein. The present technology is also not limited to use with respirator devices that create a positive pressure environment. For example, the present technology can be utilized in a negative pressure environment, such as with the respiratory devices described in New Zealand Provisional Patent Application No. 757200, filed Sep. 12, 2019, the disclosure of which is incorporated by reference herein in its entirety.

### C. Selected Embodiments for Determining Corrected Values of Measured Breath Metrics

As previously described, the present technology provides “open” respiratory devices/systems in which the mask component does not directly interface with a user's face to form a seal. Because the mask component is at least partially spaced apart from the user's mouth, the user's exhalation gases must pass through a void (e.g., the breathing chamber 108) before reaching sensors that are coupled to the mask component. Therefore, the user's exhalation gases mix with other gases in the breathing chamber (e.g., the exhalation gases mix with the filtered atmospheric air) to form a mixed gas, which can also be referred to as “diluted exhalation gases” and “chamber gases.” This mixing occurs before the exhalation gases reach the sensor. As a result, the user's exhalation gases are diluted before they reach the sensors 130.

FIGS. 2A-2B, which are top views of a user wearing the device 100, schematically illustrate the dilution of the user's exhalation gases before the exhalation gases reach the sensors 130 (only one sensor 130 is shown in FIGS. 2A and 2B). FIG. 2A depicts a stage of operation before the user exhales (e.g., during inhalation or between breaths). During this phase of the breathing cycle, the fans 114a-b direct filtered atmospheric air (e.g., shown by arrows 222) into the breathing chamber 108 to create a positive pressure environment (e.g., relative to the atmospheric air) in the breathing chamber 108 and to provide filtered air for the user to inhale. FIG. 2B depicts a second stage of operation during the exhalation phase of the breathing cycle. During this phase, the user's exhalation gases (e.g., shown by arrows 224) enter the breathing chamber 108 and mix with the filtered atmospheric air. Notably, this mixing typically occurs in the breathing chamber 108 before the exhalation gases reach the sensor 130. Therefore, as previously noted, the portion of the exhalation gases reaching the sensor 130 is diluted with filtered atmospheric air. As described below, the present technology provides techniques for accounting for the mixing of the user's exhalation gases and the filtered atmospheric air in order to provide accurate readings from the sensors 130.

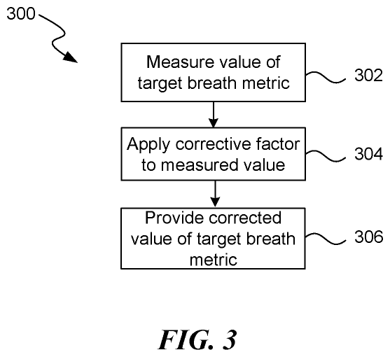

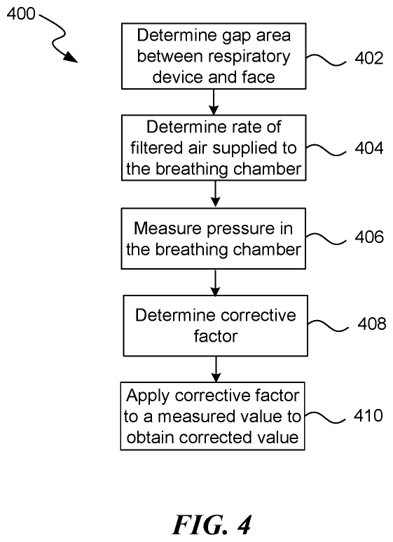

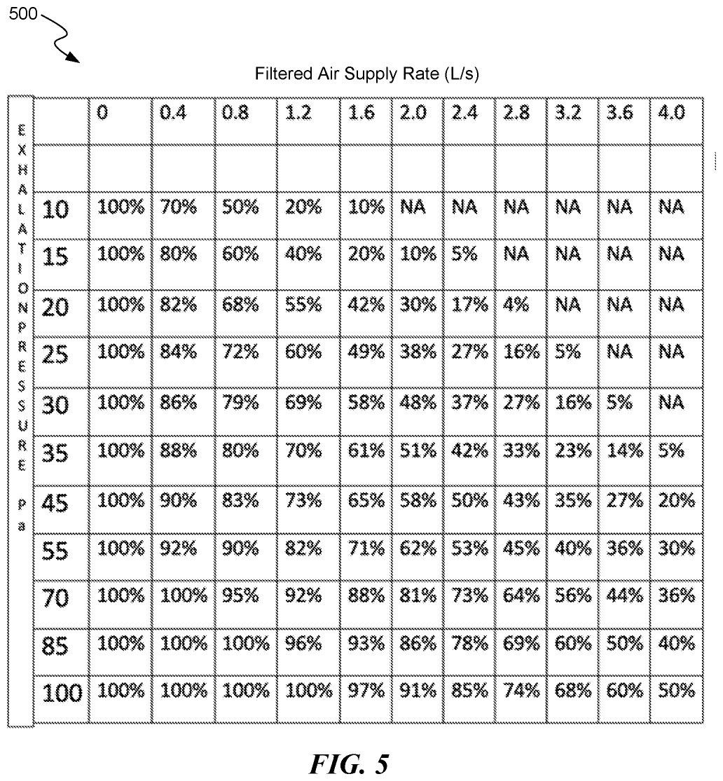

FIG. 3 is a flowchart of a method 300 for determining a corrected value of a breath metric in accordance with embodiments of the present technology. In some embodiments, method 300 can be carried out in part or in whole by the device 100 or another suitable respiratory system. Method 300 begins in step 302 by measuring a value of a target breath metric using a sensor (e.g., one of the sensors 130). The breath metric can be any breath metric described herein, such as a concentration of VOCs, O₂, CO₂, H₂, N₂, NO, NO₂, NOₓ, SOₓ, Ar, water vapor, and/or ammonia. The method 300 continues is step 304 by applying a corrective factor to the measured value of the target breath metric obtained in step 302. The corrective factor accounts for any change in value that may have occurred as a result of the exhalation gases mixing with atmospheric gases in the breathing chamber 108 before reaching the sensor 130. Additional features of the corrective factor, including methods of calculating the corrective factor and applying the corrective factor to the measured value, are described in detail with respect to FIGS. 4 and 5. Once the corrective factor has been applied to the measured value, the method 300 can continue by providing a corrected value of the target breath metric that corresponds to the actual value of the target particle/gas in the user's exhalation gases.

As noted previously, the present technology provides several techniques to account for the dilution of exhalation gases received at a respiratory device sensor spaced apart from a user's mouth (e.g., the sensor 130). For example, FIG. 4 is a flowchart of a method 400 of calculating a corrective factor for adjusting a measured value of a target breath metric in the user's exhalation breath. As used herein, the terms “corrective factor” and “dilution factor” refer to a ratio of exhalation gases versus the filtered atmospheric air in the breathing chamber 108. Although described with respect to the device 100, the method 400 can be performed using other respiratory devices.

The method 400 can include, in step 402, determining a gap area of the respiratory device. The gap area is a dimensionless and mathematically derived value that corresponds to the area between the device frame (e.g., the frame 106) and the user's face that is “open” to the external environment (e.g., the area defined by the gaps 112 through which air can leak out of the breathing chamber 108). The gap area can be calculated according to the following equation:

$$GA ( \frac{P}{TFSR} ) x FF$$

where GA=gap area, P=pressure in the breathing chamber 108 (e.g., as measured in Pascals), TFSR=total fan supply rate (e.g., as measured in L/s), and FF=fit factor, which is a dimensionless constant that is dependent on the individual shield and can be experimentally derived. Because the device 100 may move relative to the user over time (e.g., the user may adjust the device 100, etc.), in some embodiments the gap area is calculated continuously (e.g., constantly, periodically, etc.) and stored. The gap area can be determined automatically (e.g., via the respiratory analysis module 152) and/or in response to a user input.

The method 400 can continue in step 404 by determining the rate of filtered atmospheric air being supplied to the breathing chamber 108 (e.g., via the fans 114). In some embodiments, the rate is a predetermined or otherwise set rate that is known to the user/system. In some embodiments, the respiratory analysis module 152 can automatically determine the rate based on the speed of the fans 114. The rate can be between about 1 L/s and about 250 L/s, such as about 1, 2, 3, 4, 5, 6, 7, 8, 9, 10, 15, 20, 25, 50, 75, 100, 150, 200, or 250 L/s.

The method 400 can continue in step 406 by measuring the pressure in the breathing chamber 108. The pressure in the breathing chamber 108 can be measured using a conventional pressure sensor in fluid communication with the breathing chamber 108. If the gap area and the supply rate of atmospheric air remain constant, the pressure measured in the system is proportional to the user's respiration. For example, the pressure in the breathing chamber 108 decreases as the user inhales and increases as the user exhales. Without being bound by theory, the pressure in the breathing chamber is expected to be between about 2 Pa and about 150 Pa. In some embodiments, the method 400 may optionally calculate a user's respiratory rate and/or respiratory volume based on the measured pressure. For example, the respiratory analysis module 152 can measure the pressure in the breathing chamber 108 during a period in which the user is neither inhaling nor exhaling to establish a baseline pressure. With the gap area and supply rate of atmospheric air constant, a change in the pressure in the breathing chamber 108 can be used to calculate the user's respiratory rate and/or respiratory rate. For example, if the pressure increased in the system from a baseline of 10 Pa to 20 Pa, this indicates the user exhaled a volume of air at a rate sufficient to raise the pressure in the breathing chamber 108 by 10 Pa.

The method 400 can continue in step 408 by determining, based at least in part on (i) the gap area, (ii) the atmospheric air supply rate, and/or (iii) the pressure (and/or the user respiratory volume or rate, if calculated), a corrective factor. The corrective factor is generally proportional to the pressure value determined in step 406 (e.g. a relatively higher pressure in the breathing chamber 108 indicates the user is exhaling, and thus a greater percentage of the gases in the breathing chamber are exhalation gases) and inversely proportional to the atmospheric air supply rate determined in step 404 (e.g., a relatively higher supply rate means more atmospheric air is in the chamber, which dilutes the user's exhalation gases). In some embodiments, the corrective factor can be calculated automatically by the respiratory analysis module 152. For example, the respiratory analysis module 152 may mathematically derive the corrective factor based on the gap area, the atmospheric air supply rate, and the pressure.

Alternatively or additionally, the corrective factor can be determined in step 408 by using a “look-up-table” derived from experimental data based on a constant gap area. The look-up-table can be generated through a protocol comparing the user's breath biometrics measured using a sealed respiratory system with the user's breath biometrics measured using the device 100. The measurements using the device 100 can be taken at various gap areas, atmospheric air supply rates, and pressures. The difference between the values obtained using the sealed system and the values obtained using the device 100 can be used to determine the corrective factor for a given gap area, supply rate, and pressure value. The look-up-table can alternatively be derived through other suitable experimental protocols.

FIG. 5 illustrates an exemplary look-up-table 500 for a user having a gap area of 25. As illustrated, each row represents a different pressure value in the breathing chamber 108 and each column represents a filtered air supply rate provided by the fans 114. Based on the measured pressure value and filtered air supply rates (e.g., as determined in steps 406 and 404, respectively), a user and/or the respiratory analysis module 152 can determine the corrective factor by referencing the look-up-table 500. For example, if the filtered air supply rate is 1.6 L/s and the pressure in the breathing chamber 108 is 45 Pa, the corrective factor is 65%. This means that 65% of the air received at the sensor is exhalation gases. As another example, if the filtered air supply rate is 0.4 L/s and the pressure in the breathing chamber is 55 Pa, the corrective factor is 90%. This means that 90% of the received at the sensor 130 is exhalation gases.

As one skilled in the art will appreciate from the disclosure herein, the corrective factor changes depending on the stage of the breath cycle. Accordingly, the device 100 may determine the corrective factor for the time at which the target breath metric was measured. For example, the sensors 130 may measure a pressure in the chamber and/or the atmospheric air supply rate at substantially the same time as measuring the target breath metric. For example, in some embodiments, the device 100 may measure the pressure in the breathing chamber 108 continuously or semi-continuously, such as about 20 times per second, about 15 times per second, about 10 times per second, about 5 times per second, about once per second, or more or less frequently, to ensure that a pressure measurement is taken at substantially the same time as the measuring the target breath metric. The measured pressure and/or atmospheric air supply rate can then be used to calculate the corrective factor, as described above. The corrective factor can then be applied to the measured target breath metric to obtain the corrected value.

Returning to FIG. 4, with the corrective factor known, the method 400 can continue in step 410 by applying the corrective factor to a measured value of a target breath metric. For example, if the corrective factor determined in step 408 was 65%, this means that 65% of the air at the sensor 130 is exhalation gases. Therefore, the corrected value of the measured metric is 65% of the value recorded by the sensor 130. For example, if the sensor is a hydrogen sensor and recorded a value of 10 ppm during an exhalation phase of the user's breath, then the corrected level of hydrogen in the breath (after applying the corrective factor) would be 6.5 ppm, assuming there is no hydrogen in the filtered atmospheric air. In some embodiments, step 410 is performed by the respiratory analysis module 152, although step 410 can also be performed by another computing device/system, and/or manually.

In some embodiments, the respiratory analysis module 152 may receive and/or store a baseline measurement associated with the target metric's concentration in the atmospheric air (if present), and account for this baseline presence when determining the corrected value. The baseline measurement can be obtained using the sensor 130 before the user puts on the device 100 or during a non-exhalation phase of the breathing cycle. As an example, the baseline concentration of oxygen in the breathing chamber 108 can be 20%. If the corrective factor was determined to be 50%, and if the sensor 130 recorded an oxygen concentration of 10% during the exhalation phase of the user's breath, the corrected value of oxygen in the exhalation gases is 0%.

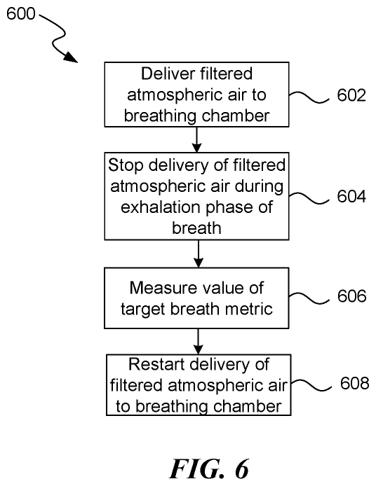

FIG. 6 is a flowchart of a method 600 for measuring breath metrics using a sensor (e.g., the sensor 130) in an open respiratory device (e.g., the device 100). However, unlike the method 400 described above, the method 600 does not need to adjust for dilution when sensing metrics associated with a user's breath. Although described with respect to the device 100, one the method 400 can be performed using other suitable respiratory devices.

The method 600 includes, in step 602, delivering atmospheric air to a breathing chamber of an open respiratory device. For example, the fans 114a-b on the device 100 can be operated to provide filtered atmospheric air to the breathing chamber 108, as previously described. The filtered air provides a positive pressure in the breathing chamber 108 to prevent unfiltered atmospheric air from entering the breathing chamber 108. The method 600 can continue in step 604 by stopping the delivery of the filtered atmospheric air to the breathing chamber 108 during an exhalation phase of a user breath. The exhalation gases therefore do not generally mix with the filtered atmospheric air, and instead arrive at the sensor 130 without being substantially diluted. The exhalation phase of the user's breath can be detected through any suitable means, such as based on the pressure in the breathing chamber. For example, the pressure within the chamber can be continuously or semi-continuously (e.g., about 10 times per second) measured using a pressure sensor. If the measured pressure exceeds a predetermined value (e.g., a baseline pressure provided by the fans 114a-b), the device 100 terminates delivery of the filtered atmospheric air to the breathing chamber 108. With the delivery of the filtered atmospheric air to the breathing chamber stopped, the method 600 continues in step 606 by measuring one or more values corresponding to one or more breath metrics.

Alternatively, in some embodiments the direction of airflow provided by the fans 114a-b is reversed during the exhalation phase of the user's breath, thereby sucking air (e.g., the filtered atmospheric air, the exhalation gases, or both) out of the breathing chamber 108. In some embodiments, the fans 114a-b may draw exhalation gases into a specific portion of the device 100 at which the sensors 130 are positioned. In some embodiments, the fans 114a-b operate to draw a predetermined amount of atmospheric air in combination with exhalation gases into the specific portion of the device 100 at which the sensors are positioned.

Regardless of whether the fans are stopped or reversed during exhalation, once the sensor records the desired breath metric(s), the method 600 can continue by restarting delivery of the filtered atmospheric air to the breathing chamber 108 (e.g., by restarting or reversing the direction of the fans 114). Without being bound by theory, it is expected that unfiltered atmospheric air will not leak into the breathing chamber 108 when the fans are turned off during the exhalation phase due to the increased pressure in the breathing chamber 108 provided by the exhalation gases.

### D. Examples

The following examples are included to further describe some aspects of the present technology, and should not be used to limit the scope of the invention.

1. A respirator system, comprising:
a mask component defining a breathing chamber, wherein the mask component is configured to fit at least partially over a user's face with a gap between a periphery of the mask component and the user's face;
a fan unit configured to deliver external air to the breathing chamber;
a sensor coupled to the mask component, wherein the sensor is configured to (i) be at least partially spaced apart from the user's mouth across the breathing chamber, and (ii) measure a breath metric associated with the user's exhalation gases; and
a respiratory analysis module storing instructions that, when executed, automatically adjust a value of the measured breath metric to provide a corrected value that accounts for a dilution of the user's exhalation gases in the breathing chamber.

2. The respirator system of example 1 wherein the instructions, when executed, cause the respiratory analysis module to:
receive an air supply rate corresponding to a rate of the external air provided to the breathing chamber by the fan unit;
receive a pressure value corresponding to a pressure in the breathing chamber; and
based at least in part on the air supply rate and the pressure value, calculate a corrective factor, wherein the corrective factor is applied to the measured breath metric to provide the corrected value.

3. The respirator system of example 2 wherein the instructions, when executed, further cause the respiratory analysis module to:
calculate, based at least in part on the received pressure value, an exhalation gas flow rate corresponding to the user's exhalation gases; and
based at least in part on the air supply rate and the exhalation gas flow rate, calculate the corrective factor.

4. The respirator system of example 2 wherein the sensor is a first sensor, the system further comprising a second sensor configured to determine the air supply rate and a third sensor configured to determine the pressure value.

5. The respirator system of any of examples 1-4 wherein the mask component includes a frame and a shield, and wherein the sensor is coupled to the frame and/or the shield.

6. The respirator system of example 5 wherein the sensor is coupled to a central portion of the frame and/or the shield positionable in front of the user's mouth.

7. The respirator system of any of examples 1-6, further comprising a housing having a valve moveable between an open position in which an interior of the housing is in fluid communication with the breathing chamber and a closed position in which the interior of the housing is fluidly isolated from the breathing chamber, wherein the sensor is positioned within the housing.

8. The respirator system of example 7 wherein the valve is configured to be in the open position during an exhalation phase of the user's breath.

9. The respirator system of example 7 or 8 wherein the valve is configured to be in the closed position during an inhalation phase of the user's breath.

10. The respirator system of any of examples 7-9 wherein the valve is configured to automatically move between the open and closed positions based on a pressure in the breathing chamber.

11. The respirator system of any of examples 1-10 wherein the breath metric is a concentration of volatile organic compounds, oxygen, carbon dioxide, hydrogen, nitrogen, nitric oxide, nitric dioxide, total nitric oxides, sulphur oxides, argon, water vapor, and/or ammonia in the user's exhalation gases.

12. The respirator system of any of examples 1-10 wherein the breath metric is a humidity level or temperature of the user's exhalation gases.

13. The respirator system of any of examples 1-12 wherein the sensor is a first sensor, the respirator device further comprising a second sensor, and wherein the second sensor is a pressure sensor, a flow sensor, a temperature sensor, an audio sensor, or a position sensor.

14. The respirator system of any of examples 1-13 wherein the fan unit is configured to maintain a positive pressure within the breathing chamber.

15. The respirator system of any of examples 1-14, further comprising a display element configured to display the corrected value of the measured metric.

16. The respirator system of any of examples 1-15, further comprising a drug delivery feature configured to deliver a drug to the user if the corrected value of the metric exceeds a first predetermined threshold and/or falls beneath a second predetermined threshold.

17. A method of determining a breath metric of a user wearing an open respiratory device having a sensor spaced apart from the user's mouth by a breathing chamber, the method comprising:
measuring, via the sensor, a value of the breath metric in chamber gases during an exhalation phase of the user's breath, wherein the chamber gases include patient exhalation gases and atmospheric air;
calculating a corrective factor, wherein the corrective factor corresponds to a ratio between the patient exhalation gases in the chamber gases and the atmospheric air in the chamber gases; and
applying the corrective factor to the measured value of the metric to determine a corrected value of the breath metric.

18. The method of example 17 wherein calculating the corrective factor includes:
determining an air supply rate of atmospheric air entering the breathing chamber;
determining a pressure in the breathing chamber; and
based at least in part on the air supply rate and the pressure in the breathing chamber, calculating the corrective factor.

19. The method of example 18, further comprising determining a gap area corresponding to a gap between the respiratory device and the user's face, wherein calculating the corrective factor is further based at least in part on the gap area.

20. The method of any of examples 17-19, further comprising displaying the corrected value of the breath metric to the user.

21. The method of any of examples 17-20, further comprising generating an alert if the corrected value of the breath metric exceeds a predetermined threshold.

22. The method of any of examples 17-21, further comprising generating an alert if the corrected value of the breath metric falls below a predetermined threshold.

23. The method of any of examples 17-22 wherein the breath metric is a concentration of volatile organic compounds, oxygen, carbon dioxide, hydrogen, nitrogen, nitric oxide, nitric dioxide, total nitric oxides, sulphur oxides, argon, water vapor, and/or ammonia in the chamber gases.

24. The method of any of examples 17-22 wherein the breath metric is a humidity level or temperature of the chamber gases.

25. A method of determining a breath metric of a user wearing an open respiratory device having a sensor spaced apart from the user's mouth by a breathing chamber, the method comprising:
delivering atmospheric air to the breathing chamber, wherein delivering the filtered air provides a positive pressure in the breathing chamber;
stopping delivery of the filtered atmospheric air to the breathing chamber during an exhalation phase of the user's breath;
measuring a value of a breath metric associated with the user's exhalation gases during the exhalation phase of the user's breath; and
after measuring the value of the breath metric, restarting delivery of the atmospheric air to the breathing chamber.

26. The method of example 25 wherein the breathing chamber is fluidly coupled to an external environment via one or more gaps between the user and the device, and wherein delivering the filtered air to the breathing chamber provides a positive pressure in the breathing chamber to prevent external air from leaking into the breathing chamber through the one or more gaps.

27. The method of example 25 or 26 wherein the breathing chamber maintains a positive pressure throughout the steps of delivering, stopping, measuring, and restarting to prevent unfiltered external air from leaking into the breathing chamber through one or more gaps between the open respiratory device and the user's face.

28. The method of any of examples 25-27 wherein the breath metric is a concentration of volatile organic compounds, oxygen, carbon dioxide, hydrogen, nitrogen, nitric oxide, nitric dioxide, total nitric oxides, sulphur oxides, argon, water vapor, and/or ammonia in the user's exhalation gases.

29. The method of any of examples 25-27 wherein the breath metric is a humidity level or temperature of the user's exhalation gases.

### E. Conclusion

The above detailed description of embodiments of the technology are not intended to be exhaustive or to limit the technology to the precise forms disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

The embodiments described above may also be said broadly to consist in the parts, elements and features referred to or indicated in the specification of the application, individually or collectively, and any or all combinations of any two or more said parts, elements or features.

Further, where specific integers are mentioned herein which have known equivalents in the art to which the embodiments relate, such known equivalents are deemed to be incorporated herein as if individually set forth.

Unless the context clearly requires otherwise, throughout the description and the examples, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. As used herein, the phrase “and/or” as in “A and/or B” refers to A alone, B alone, and A and B. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. A respirator system, comprising:
a mask component defining a breathing chamber, wherein the mask component is configured to fit at least partially over a user's face with a gap between a periphery of the mask component and the user's face;
a fan unit configured to deliver external air to the breathing chamber;
a sensor coupled to the mask component, wherein the sensor is configured to (i) be at least partially spaced apart from the user's mouth across the breathing chamber, and (ii) measure a breath metric associated with the user's exhalation gases; and
a respiratory analysis module storing instructions that, when executed, automatically adjust a value of the measured breath metric to provide a corrected value that accounts for a dilution of the user's exhalation gases in the breathing chamber.

2. The respirator system of claim 1 wherein the instructions, when executed, cause the respiratory analysis module to:
receive an air supply rate corresponding to a rate of the external air provided to the breathing chamber by the fan unit;
receive a pressure value corresponding to a pressure in the breathing chamber; and
based at least in part on the air supply rate and the pressure value, calculate a corrective factor, wherein the corrective factor is applied to the measured breath metric to provide the corrected value.

3. The respirator system of claim 2 wherein the instructions, when executed, further cause the respiratory analysis module to:
calculate, based at least in part on the received pressure value, an exhalation gas flow rate corresponding to the user's exhalation gases; and
based at least in part on the air supply rate and the exhalation gas flow rate, calculate the corrective factor.

4. The respirator system of claim 2 wherein the sensor is a first sensor, the system further comprising a second sensor configured to determine the air supply rate and a third sensor configured to determine the pressure value.

5. The respirator system of claim 1 wherein the mask component includes a frame and a shield, and wherein the sensor is coupled to the frame and/or the shield.

6. The respirator system of claim 5 wherein the sensor is coupled to a central portion of the frame and/or the shield positionable in front of the user's mouth.

7. The respirator system of claim 1, further comprising a housing having a valve moveable between an open position in which an interior of the housing is in fluid communication with the breathing chamber and a closed position in which the interior of the housing is fluidly isolated from the breathing chamber, wherein the sensor is positioned within the housing.

8. The respirator system of claim 7 wherein the valve is configured to be in the open position during an exhalation phase of the user's breath.

9. The respirator system of claim 7 wherein the valve is configured to be in the closed position during an inhalation phase of the user's breath.

10. The respirator system of claim 7 wherein the valve is configured to automatically move between the open and closed positions based on a pressure in the breathing chamber.

11. The respirator system of claim 1 wherein the breath metric is a concentration of volatile organic compounds, oxygen, carbon dioxide, hydrogen, nitrogen, nitric oxide, nitric dioxide, total nitric oxides, sulphur oxides, argon, water vapor, and/or ammonia in the user's exhalation gases.

12. The respirator system of claim 1 wherein the breath metric is a humidity level or temperature of the user's exhalation gases.

13. The respirator system of claim 1 wherein the sensor is a first sensor, the respirator device further comprising a second sensor, and wherein the second sensor is a pressure sensor, a flow sensor, a temperature sensor, an audio sensor, or a position sensor.

14. The respirator system of claim 1 wherein the fan unit is configured to maintain a positive pressure within the breathing chamber.

15. The respirator system of claim 1, further comprising a display element configured to display the corrected value of the measured metric.

16. The respirator system of claim 1, further comprising a drug delivery feature configured to deliver a drug to the user if the corrected value of the metric exceeds a first predetermined threshold and/or falls beneath a second predetermined threshold.

17. A method of determining a breath metric of a user wearing an open respiratory device having a sensor spaced apart from the user's mouth by a breathing chamber, the method comprising:
measuring, via the sensor, a value of the breath metric in chamber gases during an exhalation phase of the user's breath, wherein the chamber gases include patient exhalation gases and atmospheric air;
calculating a corrective factor, wherein the corrective factor corresponds to a ratio between the patient exhalation gases in the chamber gases and the atmospheric air in the chamber gases; and
applying the corrective factor to the measured value of the metric to determine a corrected value of the breath metric.

18. The method of claim 17 wherein calculating the corrective factor includes:
determining an air supply rate of atmospheric air entering the breathing chamber;
determining a pressure in the breathing chamber; and
based at least in part on the air supply rate and the pressure in the breathing chamber, calculating the corrective factor.

19. The method of claim 18, further comprising determining a gap area corresponding to a gap between the respiratory device and the user's face, wherein calculating the corrective factor is further based at least in part on the gap area.

20. The method of claim 17, further comprising displaying the corrected value of the breath metric to the user.

21. The method of claim 17, further comprising generating an alert if the corrected value of the breath metric exceeds a predetermined threshold.

22. The method of claim 17, further comprising generating an alert if the corrected value of the breath metric falls below a predetermined threshold.

23. The method of claim 17 wherein the breath metric is a concentration of volatile organic compounds, oxygen, carbon dioxide, hydrogen, nitrogen, nitric oxide, nitric dioxide, total nitric oxides, sulphur oxides, argon, water vapor, and/or ammonia in the chamber gases.

24. The method of claim 17 wherein the breath metric is a humidity level or temperature of the chamber gases.

25. A method of determining a breath metric of a user wearing an open respiratory device having a sensor spaced apart from the user's mouth by a breathing chamber, the method comprising:
delivering atmospheric air to the breathing chamber, wherein delivering the filtered air provides a positive pressure in the breathing chamber;
stopping delivery of the filtered atmospheric air to the breathing chamber during an exhalation phase of the user's breath;
measuring a value of a breath metric associated with the user's exhalation gases during the exhalation phase of the user's breath; and
after measuring the value of the breath metric, restarting delivery of the atmospheric air to the breathing chamber.

26. The method of claim 25 wherein the breathing chamber is fluidly coupled to an external environment via one or more gaps between the user and the device, and wherein delivering the filtered air to the breathing chamber provides a positive pressure in the breathing chamber to prevent external air from leaking into the breathing chamber through the one or more gaps.

27. The method of claim 25 wherein the breathing chamber maintains a positive pressure throughout the steps of delivering, stopping, measuring, and restarting to prevent unfiltered external air from leaking into the breathing chamber through one or more gaps between the open respiratory device and the user's face.

28. The method of claim 25 wherein the breath metric is a concentration of volatile organic compounds, oxygen, carbon dioxide, hydrogen, nitrogen, nitric oxide, nitric dioxide, total nitric oxides, sulphur oxides, argon, water vapor, and/or ammonia in the user's exhalation gases.

29. The method of claim 25 wherein the breath metric is a humidity level or temperature of the user's exhalation gases.
